(19) United States
(12) Patent Application Publication
(10) Pub. No.: US 2015/0157258 A1
(43) Pub. Date: Jun. 11, 2015

(54) METHOD AND APPARATUS FOR ASSESSMENT OF SLEEP APNEA

(71) Applicant: Oregon Health & Science University, Portland, OR (US)

(72) Inventors: Zachary Beattie, Wilsonville, OR (US); Chad Hagen, Portland, OR (US); Tamara Hayes, Portland, OR (US)

(73) Assignee: Oregon Health & Science University, Portland, OR (US)

(21) Appl. No.: 14/567,219

(22) Filed: Dec. 11, 2014

Related U.S. Application Data
(60) Provisional application No. 61/914,752, filed on Dec. 11, 2013.

(51) Int. Cl. A61B 5/00; A61B 5/113

(52) U.S. Cl. CPC A61B 5/4818; A61B 5/113; A61B 5/6891; A61B 5/746

(57) ABSTRACT

Methods and apparatuses for automatically identifying sleep apnea in a subject based on load cell signal data obtained from load cells coupled with one or more supports of a bed are disclosed. In one example approach, a method comprises continuously collecting load cell signal data from one or more load cells positioned below one or more supports of a bed, processing the signal data to obtain processed signal data, extracting features from the processed signal data, calculating a sleep apnea severity parameter based on the extracted features via a model, and identifying sleep apnea in the subject based on the sleep apnea severity parameter.

### CROSS REFERENCE TO RELATED APPLICATIONS

The present application claims priority to U.S. Provisional Patent Application No. 61/914,752, filed Dec. 11, 2013, entitled “METHOD AND APPARATUS FOR ASSESSMENT OF SLEEP APNEA,” the entire disclosure of which is hereby incorporated by reference in its entirety.

### FIELD

The present disclosure relates to the field of sleep disorder monitoring, and, more specifically, to methods and apparatuses for identifying sleep apnea in a subject during sleep.

### BACKGROUND

Disorders of sleep and wakefulness affect many people and can lead to serious health risks. For example, the Institute of Medicine has reported that 50 to 70 million Americans suffer from what they refer to as disorders of sleep and wakefulness, including more than 30 million who suffer from sleep apnea. Several serious health risks, such as cardiovascular disease, are associated with sleep apnea.

Approaches are known for monitoring sleep disorders which rely on various obtrusive sensors and subjective selection and assessment of data from the sensors to assess a subject's sleep. For example, the current standard of care for diagnosing and monitoring sleep disorders is overnight polysomnography (PSG), a multiparametric test that monitors eye movement, respiratory airflow, blood oxygen saturation, heart rhythm and other biophysical signs. However, such an approach is expensive, obtrusive, and inconvenient. For example, in such an approach, patients who are already struggling with sleep may be physically wired to several sensors and asked to sleep normally in a sleep lab. Also, these tests are not usually performed frequently enough to detect the night-to-night variance that many sleep disorders exhibit or to track a patient's progress after treatment has been prescribed. For example, based on a single night of data from a highly disruptive device, a doctor may prescribe treatment and no follow-up of the efficacy of the treatment may occur, although the patient may return to the sleep lab in 4-6 months for another evaluation.

Such sleep monitoring approaches may also rely on manual selection and subjective assessment of data received from various sensors in order to monitor and assess sleep disorders and therefore may be difficult to implement for long-term monitoring of a patient and may give rise to subjective results which vary from clinician to clinician. For example, in some approaches, a sleep technologist may visually score sensor data received from sensors during a subject's sleep. However, the sheer amount of sensor data generated during extended sleep monitoring may make it extremely difficult and time-consuming for a sleep technician to apply such a visual scoring analysis to all the data received during the sleep monitoring.

### SUMMARY

The present disclosure is directed to methods and apparatuses for automatically identifying sleep apnea in a subject based on load cell signal data obtained from load cells coupled with supports of a bed. In one example approach, a method for automatically identifying sleep apnea in a subject during sleep may comprise continuously collecting load cell signal data from one or more load cells positioned below one or more supports of a bed, processing the signal data to obtain processed signal data, extracting features from the processed signal data, calculating a sleep apnea severity parameter based on the extracted features via a model, and identifying sleep apnea in the subject based on the sleep apnea severity parameter.

In such an approach, load cells may be installed to a patient's bed to provide a non-obtrusive sleep monitoring system which may, for example, be used during in-home monitoring for an extended duration to monitor a patient's sleep patterns while they sleep in their own homes. This could allow for the triage of patients into the sleep lab, the possible diagnosis of individuals with severe sleep apnea, and/or tracking of the progress of patients over time after an initial diagnosis of sleep apnea, for example.

Such an approach provides automated processing of continuous load cell recordings to assess sleep behavior of a subject based on signal data obtained from unobtrusive load cells installed beneath the subject's bed. This approach does not rely on predefined segments of data or subjective assessments, such as visual scoring, by clinicians thereby providing automatically obtained and accurate quantitative parameters representing the severity of sleep apnea exhibited by the patient. These parameters may be used to identify sleep apnea occurrences so that actions can be taken. For example, the subject or a physician may be informed as to whether the subject exhibits apnea conditions or whether a treatment for sleep apnea is working.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 shows a block diagram of an apparatus for monitoring sleep disorders in accordance with the disclosure.

FIG. 2 shows example graphs comparing an amount of movement detected in a patient without sleep apnea with an amount of movement detected in a patient with sleep apnea.

FIG. 3 shows example graphs illustrating a method for estimating breathing amplitude from load cell signal data.

FIG. 4 shows example graphs comparing an amount of variance in breathing amplitude for a patient without sleep apnea with an amount of variance in breathing amplitude for a patient with sleep apnea.

FIG. 5 shows an example graph of apnea events in load cell breathing signal data.

FIG. 6 shows example graphs comparing a load cell's ability to detect sleep apnea versus a ground truth reference.

FIG. 7 shows example Bland-Altman plots for visualizing how well load cell breathing signals could be used to detect sleep apnea as compared to a ground truth reference.

FIG. 8 shows example receiver operating characteristic curves for illustrating how well load cell data could be used to automatically detect sleep apnea.

FIG. 9 shows an example method for automatically identifying sleep apnea in accordance with the disclosure.

FIG. 10 schematically shows an example computing system in accordance with the disclosure.

### DETAILED DESCRIPTION

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use the terms “embodiment” or “embodiments,” which may each refer to one or more of the same or different embodiments. Furthermore, the terms “comprising,” “including,” “having,” and the like, as used with respect to embodiments, are synonymous.

The description may use the terms “bed” and “bed support.” The term “bed” may be used to mean any structure with a generally horizontal surface used/intended for use in supporting a body during a period of rest and/or sleep, including (but not limited to) beds, mattresses, futons, couches, bassinets, cribs, cots, cradles, recliners, and other similar structures. “Bed support” may be used to mean any structure that physically supports a “bed” on a surface such as a floor. A bed support may be an integral part of a bed and/or may be a separate component that is added to the bed. A bed may include one, two, three, four, five, six, seven, eight or more bed supports.

The term “load cell” may be used to mean any mechanism that translates/converts force into a signal such as an electrical or analog signal. Load cells/transducers are known in the art, and the description provided herein is intended to embrace all such mechanisms. Load cells in accordance with various embodiments may be coupled to a computing device by a physical connection such as a cable or a wire, and/or may be in wireless communication with a computing device and/or another load cell.

As remarked above, approaches are known for monitoring sleep disorders which rely on various obtrusive sensors and subjective selection and assessment of data from the sensors to assess a subject's sleep. For example, the gold standard for diagnosing sleep problems is overnight polysomnography (PSG), an obtrusive test in which patients spend a night in a sleep lab wired to up to many different devices for measuring airflow, movement, electrical brain signals, etc. Such an approach is expensive, inconvenient, and time-consuming, and it interferes with normal sleep patterns. Further, some sleep monitoring approaches may rely on manual selection and subjective assessment of data received from various sensors in order to monitor and assess sleep disorders and therefore may be difficult to implement for long-term monitoring of a patient and may give rise to subjective results which vary from clinician to clinician.

Previous attempts to automatically detect sleep apnea using non-contact sensors have been made. For example, some approaches have used a radio-frequency sensor placed on a night table near the bed [e.g., see A. Zaffaroni, P. de Chazal, C. Heneghan, P. Boyle, P. Ronayne, and W. T. McNicholas, “SleepMinder: an innovative contact-free device for the estimation of the apnoea-hypopnoea index,” Conf Proc IEEE Eng Med Biol Soc, pp. 7091-4, 2009]. As another example approach, a sheet with an array of pressure sensors placed on top of the mattress is used [e.g., see T. Agatsuma, K. Fujimoto, Y. Komatsu, K. Urushihata, T. Honda, T. Tsukahara, and T. Nomiyama, “A novel device (SD-101) with high accuracy for screening sleep apnoea-hypopnoea syndrome,” Respirology, vol. 14, pp. 1143-50, 2009]. These approaches achieved good agreement between their estimates of an apnea-hypopnea index (AHI) for several patients and the AHI calculated using traditionally scored polysomnography (PSG) data. However, such approaches have limitations. For example, it is unknown how well a radio-frequency sensor will work if its “view” of the patient is occluded by items placed on the night stand or by the patient sleeping under various layers of blankets and bedding. As another example, a sheet of pressure sensors placed between the patient and their mattress may lead to discomfort especially if several nights of data are to be collected for long term monitoring. In contrast, by using non-obtrusive load cells placed under supports of a bed as described herein, data may be collected without needing to alter the sleeping environment while having a minimal risk of being altered by patients during common practices such as changing bedding or placing items on their nightstand.

Embodiments herein provide a simpler, more cost-effective way to triage sleep disorders in the general population by automatically processing signal data received from load cells coupled to or positioned beneath supports of a bed. A significantly better understanding of an individual's sleep and changes in their sleep patterns over time may be obtained by monitoring their sleep in a non-invasive manner, preferably in their own home. Furthermore, treatment may be assessed and optimized if data is available on the time course of improvements as a result of the treatment.

Embodiments herein may utilize only load cell data to automatically detect sleep apnea without relying on any manually selected segments of normal breathing and apneic breathing and without relying on subjective assessments of data by clinicians. As described in the examples below, embodiments herein may provide more reliable techniques to estimate a load cell breathing signal (CoP) and detect individual breaths allowing for more accurate breathing amplitude calculations. Such an approach may be used to automatically detect sleep apnea conditions from continuous load cell recordings during an extended sleep monitoring duration, e.g., across an entire night, that do not rely on predefined segments of data. For example, as described in the examples given below, load cell data collected while an individual sleeps may be automatically processed, e.g., via a computing device, to produce a sleep apnea severity parameter such as an apnea-hypopnea index (AHI) representing the severity of sleep apnea exhibited by the patient.

As remarked above, load cells may be coupled to or placed beneath or between one or more supports of a bed and the floor in order to obtain data non-obtrusively with potential applications to long-term in-home sleep monitoring. Such load cells may be used to detect and classify movements of a subject in bed and to assess sleep hygiene. In an embodiment, load cells placed under each support of a bed offer a unique opportunity to continuously and unobtrusively monitor patients while they sleep. The patterns of changing pressure at each support may be analyzed and inferences about various sleep parameters may be made. For example, each pressure signal from the load cells may contain information about the amplitude and variability of the person's heart rate and respiration, as well as about the number, timing, and intensity of movements. This information may be extracted from the signal in a variety of ways, e.g., by using combined time domain and frequency domain techniques, including but not limited to Fourier analysis, wavelet analysis, and/or peak detection. In some embodiments this information may be extracted from individual load cells and combined using averaging or voting techniques. In some embodiments the signals from multiple load cells may be used to determine the center of pressure on the bed and the resultant center of pressure signal may itself be used to extract the information. In some embodiments a single load cell may provide sufficient data to derive the measures of interest, including respiration, heart rate, and movement, particularly when the load cell is correctly tensioned. Data from the load cells may be collected in a person's home, allowing physicians and researchers the ability to monitor a patient's sleep over time without imposing on the patient or his/her sleep.

Embodiments described herein provide systems, apparatuses and methods for the monitoring of sleep disorders. Embodiments described herein may be adapted for use in home environments. Embodiments described herein are directed to methods and apparatuses for automatically identifying sleep apnea in a subject based on load cell signal data obtained from load cells coupled to supports of a bed, e.g., positioned below one or more supports of a bed. In one example approach, a method for automatically identifying sleep apnea in a subject during sleep may comprise continuously collecting load cell signal data from one or more load cells positioned below one or more supports of a bed, processing the signal data to obtain processed signal data, extracting features from the processed signal data, calculating a sleep apnea severity parameter based on the extracted features via a model, and identifying sleep apnea in the subject based on the sleep apnea severity parameter.

Embodiments described herein provide apparatuses for monitoring sleep. An apparatus in accordance with various embodiments may comprise: one or more load cells configured for placement below one or more supports of a bed such that the bed and the one or more bed supports are physically supported by the load cells, the load cells further configured to convert force to an electrical signal indicative of the force, and a computing device coupled to at least one of the one or more load cells, where the computing device comprises computer executable instructions for receiving signals from at least one of the one or more load cells, processing the signals to obtain signals representing periods of movement and signals representing periods of stillness, extracting features from the signals representing periods of movement and the signals representing periods of stillness, calculating a sleep apnea severity parameter based on the extracted features via a model, and identifying sleep apnea in the subject based on the sleep apnea severity parameter. In some examples, the computer executable instructions may comprise one or more pattern recognition algorithms and/or linear regression algorithms used to calculate a sleep apnea severity parameter based on features extracted from load cell signal data. In some embodiments, an apparatus for monitoring sleep may further include a transceiver coupled to at least one of the computing device and the load cells. In an embodiment further comprising an alarm, the computer executable instructions may be operable to actuate the alarm in response to identifying an incident of abnormal respiration or a sleep apnea condition where a subject has stopped breathing for a time longer than a predetermined duration, e.g., during a long central apnea.

In embodiments, one or more load cells may be coupled to or placed beneath or between supports of a bed to unobtrusively monitor subjects while they sleep. Patterns of changing pressure at each support may be analyzed and inferences about various sleep parameters may be made. In some examples, data from the load cells may be collected in a person's home, and collected data may be processed to extract information about the person's respiration, heart rate, periodic leg movement (PLM), and/or other physiological parameters. Such information may be used in the diagnosis of a sleep disorder and/or to monitor a sleep disorder or an associated treatment. Systems in accordance with embodiments may also be used by physicians and/or others to monitor a patient's/subject's sleep in hospitals, laboratories, and/or health facilities. Systems and methods may also be used to monitor sleep over time without imposing on the subject's sleep.

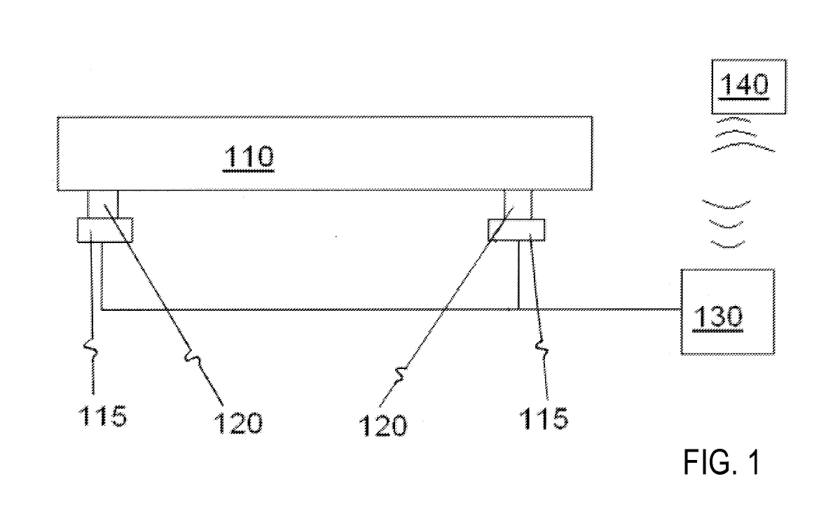

FIG. 1 illustrates a block diagram of an apparatus for monitoring sleep disorders in accordance with various embodiments. In embodiments, a bed 110 may be coupled to bed supports 120 such that the bed is physically supported by the bed supports. Load cells 115 may be positioned beneath the bed supports 120 such that the bed and bed supports are physically supported by the load cells. While two cells are shown in FIG. 1, embodiments may vary as to the number of load cells used. In some embodiments, a load cell may be placed under each corner/support of a bed and/or under one or more other bed supports. In some embodiments an additional load cell may be attached to a new support that is placed under the bed and correctly tensioned to provide support and measure the load at a specific location. The load cells 115 may be coupled to a computing device 130 comprising executable instructions for collecting data from load cells, processing the data, extracting features from the data, and detecting a sleep disorder and/or a movement associated with a sleep disorder. The computing device 130 may be in communication with an external computing device 140. Load cells 115, computing device 130 and external computing device 140 may be coupled with a physical connection such as a cable and/or wirelessly coupled, and/or may communicate with one another by means of telephony and/or telemetry.

Though FIG. 1 shows the load cells being placed below bed supports, the load cells may be coupled with or connected to components of the bed in any suitable manner. For example, load cells may be placed into, positioned between, or integrated with one or more supports of the bed, e.g., load cells may be placed on the bed frame where the box springs of the bed are supported. In particular, load cells may be coupled to or placed between any suitable components of the bed in order to measure force changes across the bed. For example, in hospital beds load cells may be placed between the components that connect the “sleeping platform” to the “bed frame.”

In embodiments, a computing device 130 may be adapted to send data to external computing device 140 to communicate information about a subject's sleep. Computing device 130 may include a personal computer, a handheld computing device, a wireless communication device, or any computing apparatus known in the art, and may be located near the bed or in another location. External computing device 140 may be a remote computing device located in another location such as a medical office, hospital, caretaker's residence, laboratory, etc. One or both of computing device 130 and/or external computing device 140 may be equipped with an alarm and logic to activate an alarm in response to an indication of a sleep disturbance/abnormal movement.

Systems and methods in accordance with various embodiments may provide for the detection of sleep disturbances by a computing device programmed with executable code operable to process signals from load cells coupled to the computing device. In some methods, one or more steps may be performed automatically by a computing device. In an embodiment, all steps of a method may be performed automatically by a computing device. Processing of signals may include filtering and/or decimating a portion of load cell signal data. In embodiments, one or more algorithms may be applied to signal data (and/or to data produced by signal data processing) to identify/differentiate between movements during sleep that are associated with respiration, PLM, and/or cardiac activity of a subject.

In some examples, after the load cells are installed to, beneath, or between supports of the bed, a calibration step may be performed on the bed/load cell system in order to characterize the response of the bed to movement so that processing of the load cell data may be adjusted and interpreted accordingly. For example, one or more impulses may be applied to the bed, e.g., by applying one or more predetermined weights to the bed, in order to characterize the bed system by determine an amount of damping and a frequency response of the system, for example. Such impulse parameters may be used during signal processing performed on the load cell signal data during sleep monitoring.

The example discussed below demonstrates the use of load cells to detect sleep apnea during sleep in a home environment, in accordance with various embodiments. In this example, data were collected from load cells placed under beds of subjects to be monitored during sleep. Features were extracted from load cell signals and used to calculate a sleep apnea severity parameter based on a model. Embodiments may vary as to the methods of signal processing, methods of feature extraction, and models used, as well as the training of the models. The example discussed below is for illustrative purposes only and is not intended to be limiting.

In this example, subjects were recruited from the Oregon Health & Science University (OHSU) sleep lab and the Pacific Sleep Program (PSP) sleep lab. Fifteen patients from the OHSU sleep lab participated in the study (an IRB was deemed unnecessary by the OHSU Institutional Review Board). Eighty-nine patients from the Pacific Sleep Program sleep lab gave informed written consent to the study (OHSU Institutional Review Board eIRB 6308). Forty-five subjects were female and 59 were male. The average age was 49.3±14.0 years and the average BMI was 32.8±7.1 kg/m².

Load cell data from the OHSU sleep lab was collected from load cells placed under each of the six supports of a king sized bed. At the PSP sleep lab, load cell data was collected from load cells that were placed under each of the five supports of a queen sized bed. At each sleep lab the load cell data was collected simultaneously with the overnight PSG data for each patient during their regularly scheduled sleep test.

For overnight sleep studies at both the OHSU and PSP sleep labs, the PSG data was scored by an experienced polysomnographic technologist employed at the corresponding sleep lab. In both cases, apneic events were scored in accordance with current American Academy of Sleep Medicine (AASM) guidelines. Apneas were scored when there was an amplitude reduction of 90% or greater for at least 10 seconds in the PSG breathing signals, and hypopneas were scored when there was an amplitude reduction of 30% in the PSG breathing signal that lasted for at least 10 seconds and was associated with at least a 4% oxygen desaturation as measured by a pulse oximeter during the PSG test. The severity of sleep apnea presented by each patient was gauged using the apnea-hypopnea index (AHI). The sum of scored apneas and hypopneas were divided by total sleep time to generate an apnea-hypopnea index from polysomnography (AHI-PSG). In addition to scoring apneic events, the technologist also scored respiratory effort related arousals (RERAs) defined by discernible reductions in airflow associated with arousal, i.e., patient awakenings, that did not meet criteria for other events. The total number of these events were combined with the sum of apneas and hypopneas and divided by total sleep time to obtain a respiratory disturbance index from polysomnography (RDI-PSG).

Several steps were involved to develop the algorithm used to automatically calculate the severity of sleep apnea, i.e., an apnea-hypopnea index automatically calculated from load cell data (AHI-LC_{AUTO}) and a respiratory disturbance index automatically calculated from load cell data (RDTLC_{AUTO}), using only the load cell data collected as a patient slept overnight on a bed with load cells placed under each support. First, the load cell data was conditioned or prepared so that relevant information about the load cell breathing signal could be extracted. Then features from the load cell breathing signal were estimated. Finally, a linear model used to combine the various load cell features into a prediction of sleep apnea severity (i.e. AHI-LC_{AUTO} and RDI-LC_{AUTO}) was trained and tested using the corresponding clinically estimated AHI-PSG and RDI-PSG.

The load cell data collected from each overnight sleep test were first trimmed to only include the period from the time the patient fell asleep to the time when the lights were turned on indicating the end of the sleep test. The low pass filtered center of pressure (CoP_{y}) signal along with the corresponding peaks and troughs of CoP_{y} representing the transitions from inspiration to expiration were then derived from the load cell data using an algorithm to automatically detect peaks and troughs in the load cell breathing signal that represent the transitions between inspiration and expiration. In particular, the developed algorithm detected all local maximums and minimums in the load cell breathing signal and eliminated any extraneous peaks and troughs that were not representative of inspiration/expiration transitions. In the algorithm, the data from each load cell was used to calculate a center of pressure along the head-to-toe axis of the bed.

Movements from the overnight load cell data were automatically detected by summing together the output from each load cell (LCₛᵤₘ) at a load cell sampling rate of 10 Hz. However, it should be appreciated that any suitable sampling rate may be used. Periods when the patient was estimated to be out of the bed, e.g., visiting the restroom, were grouped with the segments estimated to be movement and were subsequently removed. Out-of-bed segments were calculated using a K-means unsupervised clustering technique. Assuming that the peaks and troughs of the CoP_{y} breathing signal represent the maximum (peaks) and minimum (troughs) displacement of the mass moved during a breathing cycle, the breathing amplitude was estimated by calculating the difference between the peaks and troughs in the CoP_{y} signal.

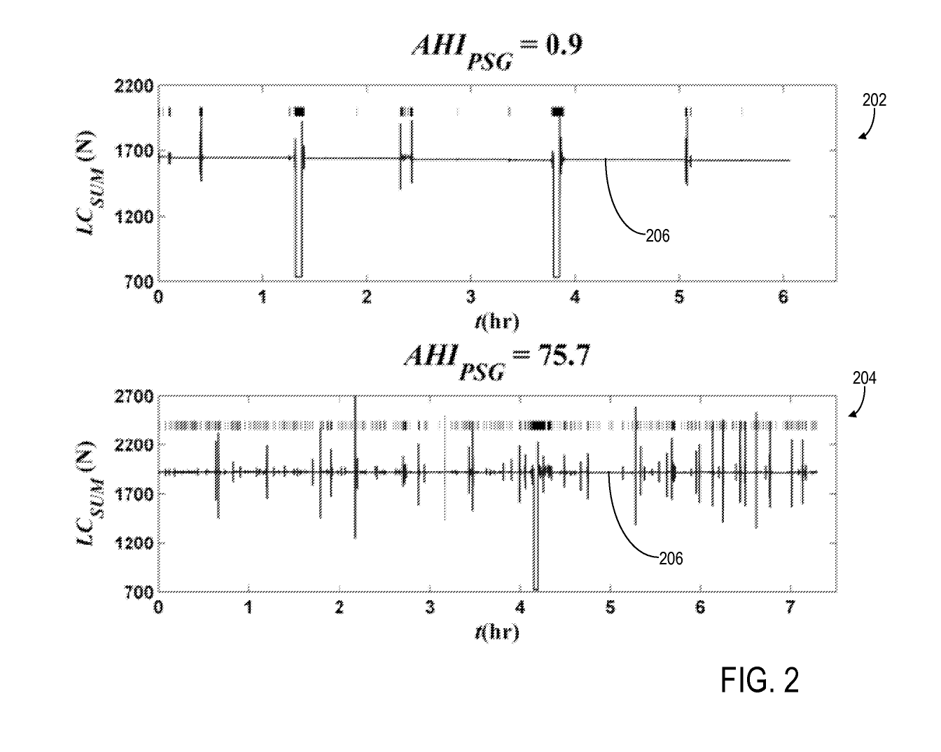

Three features were calculated from each night of load cell data. The first feature was selected to represent the overall amount of patient movement detected during the night. The assumption employed was that individuals with sleep apnea tend to have restless sleep. For example, FIG. 2 shows load cell data collected during an overnight sleep test for a patient without sleep apnea in graph 202 and a patient with severe sleep apnea in graph 204. The summed output from each load cell placed under the bed (LC_{SUM}) is shown in the trace labeled 206 in graphs 202 and 204. Movements detected using LC_{SUM} are illustrated in FIG. 2 above the load cell data traces 206. As illustrated in FIG. 2, significantly more movement was present for the patient with sleep apnea which is likely caused by small awakenings that frequently follow apneic events. This first feature is a movement index (MI) calculated using the following Equation 1:

$$MI = \frac{\# Movements}{{T}_{LC}}$$

In Equation 1, #Movements is the number of detected movements and/or out of bed segments and T_{LC} is the number of hours of load cell data collection.

Additional features were selected based on the recognition that the load cell breathing signal is tracking the movement mass caused by the diaphragm during breathing. In particular, it was recognized that when the airway into the lungs is occluded during an apneic event, the diaphragm is now pulling against a more negative pressure inside the lungs and subsequently will move less. This may lead to a gradual or sudden decrease in the amplitude of the load cell breathing signal. Eventually, as the patient increases their breathing effort during the apneic event, the diaphragm may increasingly begin to displace the mass tracked by the load cells until the apneic event is terminated. It is also possible that once the airway is open there may be an increase in breathing amplitude above normal due to the recently heightened breathing effort. The second and third features were selected to capture these constant breathing amplitude changes that are hypothesized to frequently occur in individuals with sleep apnea.

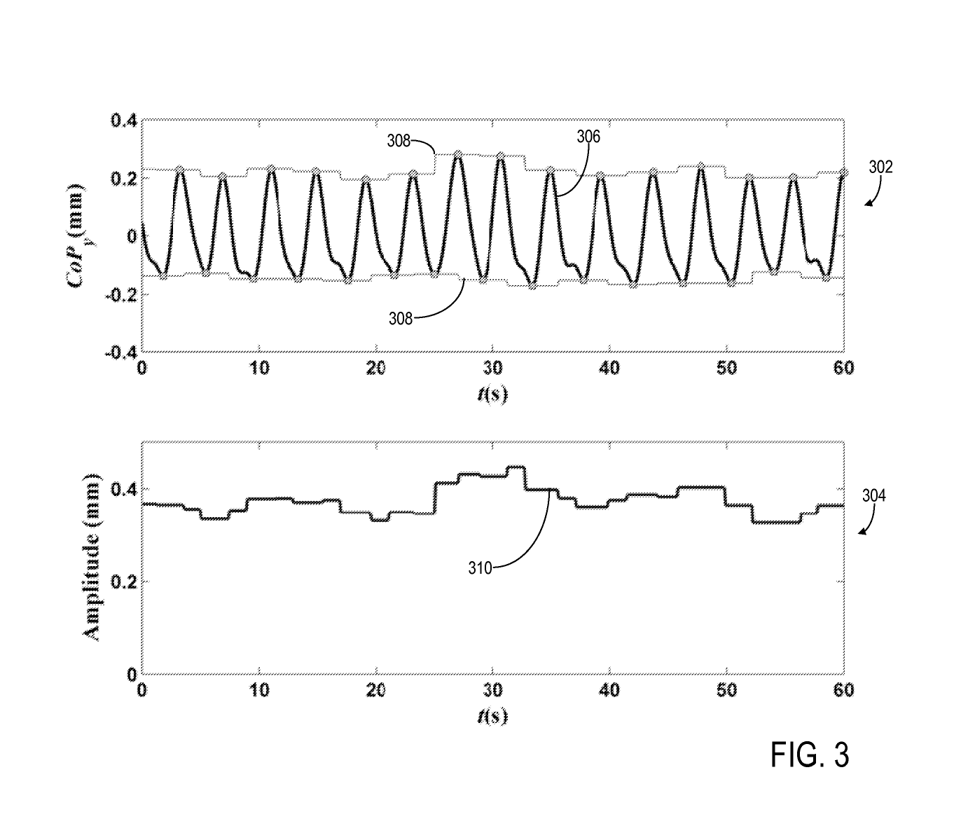

In particular, the second feature was selected to capture variance in the breathing amplitude. An example method used for estimating the breathing amplitude on a sample-by-sample basis from the load cell CoP_{y} signal for this second feature is illustrated in FIG. 3. In graph 302 in FIG. 3, the load cell breathing signal (CoP_{y}) is shown as the trace labeled 306 with detected peaks (circles) and detected troughs (squares). A peak and trough value was estimated for each individual data point in the CoP_{y} signal (traces labeled 308) using nearest neighbor interpolation from the actually detected peaks and troughs (circles and squares). In graph 304 of FIG. 3, the breathing amplitude (trace labeled 310) was estimated for every sample or data point in the CoP_{y} breathing signal by subtracting the interpolated trough values from the interpolated peak values. The variance in the load cell breathing amplitude across the entire night was estimated using the coefficient of variation (cV) for non-overlapping windows via the following Equation 2:

$$cV ( j ) = \frac{\sqrt{\frac{1}{n - 1} {\sum }_{i = 1}^{n} {( {LC}_{AMP}^{i} - {\overline{LC}}_{AMP} )}^{2}}}{\frac{1}{n} {\sum }_{i = 1}^{n} {LC}_{AMP}^{i}}$$

In Equation 2, cV(j) is the coefficient of variation for the j^{th} window, LC_{AMP} is the amplitude of the load cell breathing signal, and n is the number of amplitude estimates contained in the j^{th} window.

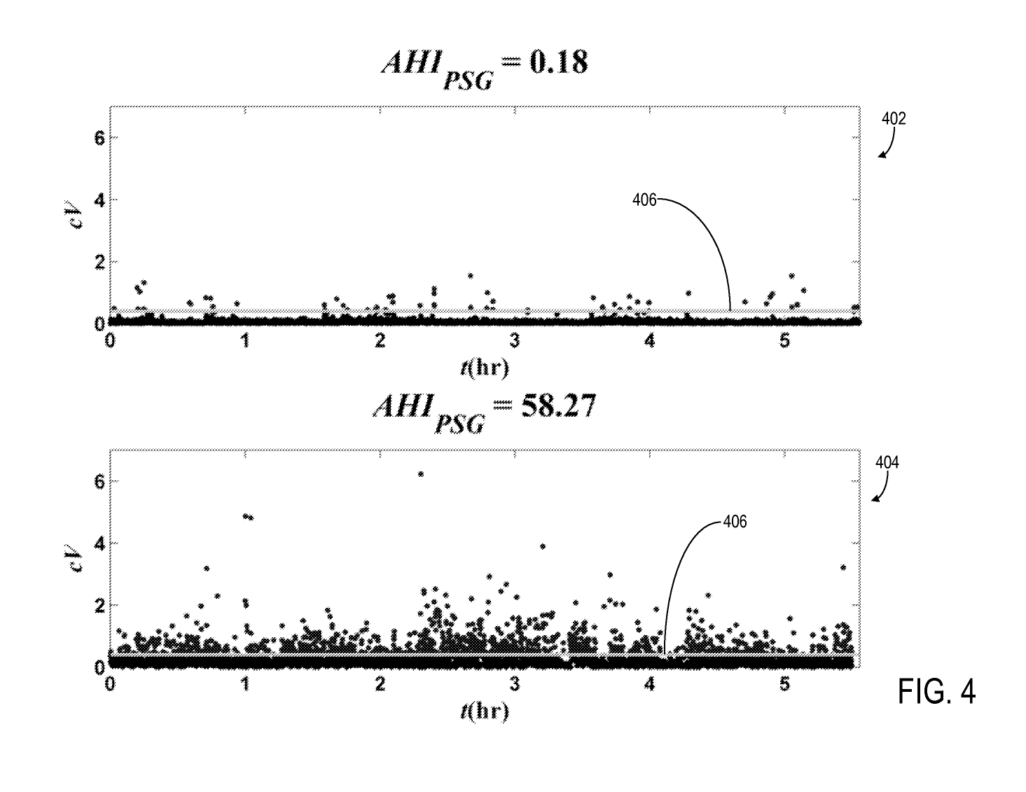

FIG. 4 show graphs of the coefficient of variation (cV) calculated from non-overlapping five second windows of load cell derived breathing amplitude. The load cell data was collected during an overnight sleep study for an individual without sleep apnea (shown in graph 402) and a patient with sleep apnea (shown in graph 404). The horizontal lines labeled 406 in graphs 402 and 404 represent a threshold of 0.4. However, it should be appreciated that any suitable threshold may be used. As illustrated in FIG. 4, the patient with sleep apnea exhibits many more segments of high breathing amplitude variability than the patient without sleep apnea. This indicates that the coefficient of variation (cV) may be higher in individuals with sleep apnea and the second feature (cV %) is defined as the ratio of time that the cV of the load cell signal is above a defined threshold (thr_{cV}) where cV % is calculated via the following Equation 3:

$$cV \% = \frac{( \# cV ( j ) > {thr}_{cV} ) {wn}_{cV}}{{t}_{LC}}$$

In Equation 3, wn_{cV} is the window size in seconds used to calculate each cV(j) and t_{LC} is the total recording time in seconds.

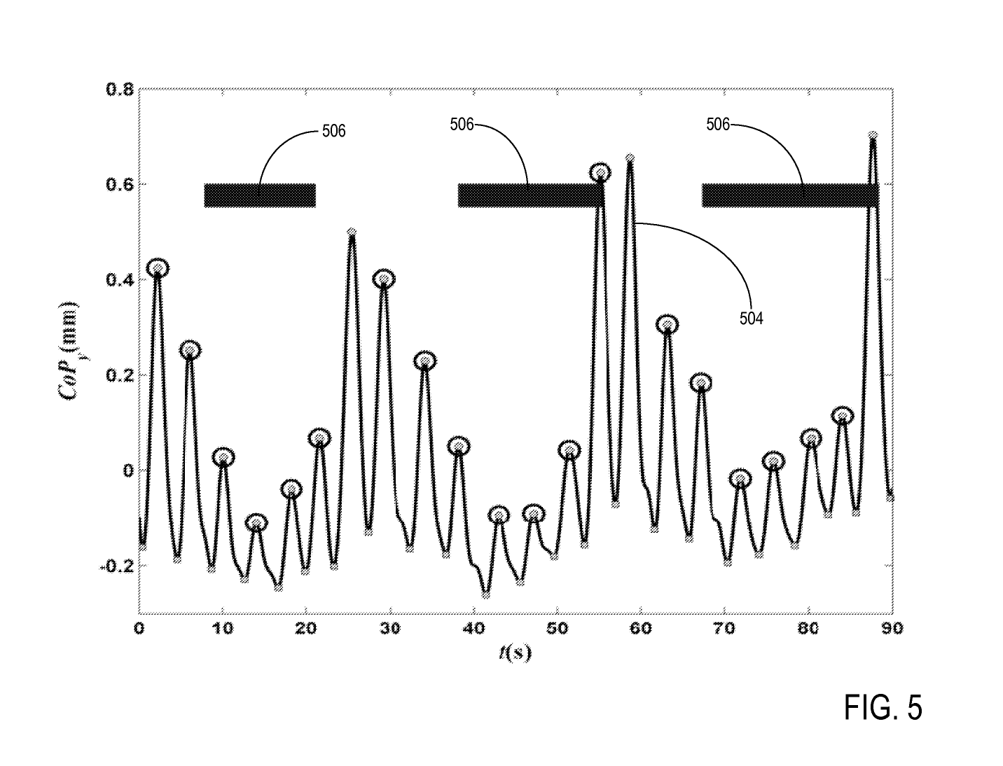

The third feature was selected to estimate the number of times per hour that the amplitude of the load cell breathing signal, i.e., CoP_{y}, decreased significantly during the course of the overnight sleep study. This third feature was also used to capture the continual decreasing and increasing of load cell breathing amplitude that is often observed during apneic events. For example, FIG. 5 shows a graph of ninety seconds of load cell breathing signal (trace labeled 504) collected from a patient during an overnight sleep study illustrating the decreasing followed by increasing breathing amplitude often observed in the load cell data during apneic/hypopneic events. In FIG. 5, automatically detected peaks and troughs used for estimating the breathing amplitude on a breath-by-breath basis are displayed as circles and squares, respectively. The peaks of breaths automatically determined to be part of a disordered breathing event are encased in circles in FIG. 5. The peak at approximately 55 seconds in FIG. 5 appears to represent a breath of significant amplitude. This peak was considered a continuation of the crescendo effect at the end of the disordered breathing event due to its amplitude—estimated as the difference between the peak value and the following trough value—being less than the following breath's amplitude at approximately 60 seconds estimated the same way. As a reference, time periods that were visually scored as apneas/hypopneas by a sleep technologist using a load cell (LC) scoring montage are presented as horizontal lines labeled 506 in FIG. 5.

In order to calculate the third feature, the amplitude of the load cell breathing signal was estimated on a breath-by-breath basis using the difference between peak/trough pairs in the load cell CoP_{y} signal. The amplitude of each breath was calculated twice—once using the difference between the peak value of the breath and the following trough value and once using the difference between the same peak value of the breath and the previous trough value. The methodology described in the following utilized both amplitude estimates independently to detect disordered breathing events and then combined the resulting disordered breathing events found using both estimates.

Disordered breathing events, e.g., apneas or hypopneas, were identified by first locating and marking/flagging individual breaths that had amplitudes that were less than a defined percentage (AMP %) of the median breathing amplitude that was estimated over the previous N seconds. Second, the breathing amplitudes of the individual breaths directly before any of these marked/flagged breaths were searched for a decrescendo effect in the breathing amplitudes. In other words, any breath that immediately preceded the originally marked breath that had a breathing amplitude less than the breath directly before it was marked/flagged as part of the disordered breathing event. Then, in a similar manner, the breathing amplitudes of the individual breaths directly after any of the originally marked breaths were searched for a crescendo effect in the breathing amplitudes, i.e., any breath with an amplitude larger than the breath directly after it was included in the disordered breathing event. Finally, the third feature is a disordered breathing index (DBI) calculated using the following Equation 4:

$$D B I = \frac{\# {Breath}_{Disordered}}{{T}_{LC}}$$

In Equation 4, #Breath_{Disordered} is the number of disordered breathing events identified that met a minimum time duration constraint (tₐₚₙₑₐ) and T_{LC} is the number of hours of collected apnea, load cell data after movement and/or out of bed periods have been removed.

The three features selected were utilized to automatically estimate sleep apnea severity, i.e., AHI-LC_{AUTO}, using a linear model with constant coefficients β₁, β₂, β₃, and β₄ via the following Equation 5:

AHI_{LC}_{_{AUTO}}=β₁+β₂(MI)+β₃(cV %)+β₄(DBI)

A leave one out method was used to iteratively train and test the model. For each iteration, features calculated from the load cell data for one patient were held out. Then linear regression was used to estimate the model coefficients β₁, β₂, β₃, and β₄ by fitting the features from the remaining 103 patients to their corresponding AHI-PSG in a least-squares sense. The AHI-LC_{AUTO} was estimated for the patient whose data was held out using these model coefficients and the features estimated for this patient. The whole process was repeated to estimate an AHI-LC_{AUTO} for each of the 104 patients. An RDI-LC_{AUTO} was also predicted for each of the 104 patients in the same manner with the exception that the model coefficients β₁, β₂, β₃, and β₄ were estimated utilizing RDI-PSG.

For the cV % and DBI features various thresholds and window sizes were initially unknown. In order to maximize the effectiveness of the cV % feature, the threshold (thr_{cV}) for distinguishing high variability from low variability and the size (in seconds) of the non-overlapping windows (wn_{cV}) was selected. For the DBI feature, the N previous seconds used to calculate the median breathing amplitude reference and the percentage of this reference amplitude (AMP %) that indicated a significant breathing amplitude attenuation was also selected. Also, the minimum time duration (in seconds) of disordered breathing segments (tₐₚₙₑₐ) needed for the segment to be considered an apnea or hypopnea was selected. In order to select suitable values for these parameters, the following ranges of values for each parameter was investigated: thr_{cV}=[0.1, 0.2, 0.3, 0.4, 0.5, 0.6, 0.7, 0.8, 0.9, 1], wn_{cV}=[5, 10, 20, 30, 40, 50, 60, 70, 80, 90, 120], N=[30, 60, 90, 120], AMP %=[10, 30, 50, 70, 90], and tₐₚₙₑₐ=[5, 10, 15, 20]. An exhaustive search was carried out across every possible combination of these parameters in order to discover the combination that optimized the estimation of AHI-LC_{AUTO} and RDI-LC_{AUTO}. In some examples, a gradient descent method may be used to search across a larger space for the optimal combination of parameters (i.e., thr_{cV}, wn_{CV}, N, AMP %, and tₐₚₙₑₐ) that maximize the ability of cV % and DBI to help estimate AHI-LC_{AUTO} or RDI-LC_{AUTO}. Such an approach may be used to identify values for these parameters that enhance the ability—measured using some combination of R² and mse, for example—of the linear models used to estimate AHI-LC_{AUTO} or RDI-LC_{AUTO}. Parameter values could also be selected that lead to accurate linear models with more intuitive coefficients.

The combination of parameters (i.e., thr_{cV}, wn_{cV}, N, AMP %, and tₐₚₙₑₐ) that resulted in the highest coefficient of determination (R²) and lowest mean squared error (mse) were selected when using AHI-PSG as the ground truth reference. R² was calculated using the following Equation 6:

$${R}^{2} = \frac{\sum {( {AHI}_{PSG} - {AHI}_{{LC}_{AUTO}} )}^{2}}{\sum {( {AHI}_{PSG} - {\overline{AHI}}_{PSG} )}^{2}}$$

In Equation 6, A̅H̅I̅_{PSG} is the mean of AHI-PSG, and mse was calculated using the following Equation 7:

$$mse = \frac{1}{n - 4} \sum {( {AHI}_{{LC}_{AUTO}} - {AHI}_{PSG} )}^{2}$$

In Equation 7, (n−4) is the number of AHI-LC_{AUTO} minus the number of coefficients in the linear model shown in Equation 5. The same method for choosing optimal parameters was utilized when using RDI-PSG as a ground truth method.

Comparison of AHI-PSG and RDI-PSG to AHI-LC_{AUTO} and RDI-LC_{AUTO} respectively was analyzed using paired t-tests with 95% confidence intervals for the difference between the two scorings. Bland-Altman plots were used to visually compare the results. Finally, the ability of the automatic scoring algorithm using the load cell data to detect sleep apnea was assessed using receiver operating characteristic (ROC) curves. The sensitivities and specificities for detecting sleep apnea using AHI-LC_{AUTO} at various thresholds were calculated. The following three AHI cutoff levels were considered for defining overnight sleep tests as being positive for sleep apnea: AHI-PSG≧5, AHI-PSG≧15, and AHI-PSG≧30. The sensitivities and specificities for detecting sleep apnea using RDI-PSG_{AUTO} were also calculated using the following three respiratory disturbance index (RDI) cutoff levels for positive sleep apnea tests: RDI-PSG≧15, RDI-PSG≧30, and RDI-PSG≧60.

The optimal combination of parameters when comparing the automatic scoring algorithm to AHI-PSG was thr_{cV}=0.4, wn_{cV}=5 s, N=30 s, AMP %=50%, and tₐₚₙₑₐ=10 s. The optimal combination when comparing to RDI-PSG was thr_{cV}=0.7, wn_{cV}=5 s, N=120 s, AMP %=50%, and tₐₚₙₑₐ=10 s. Tables 1 and 2 list the average linear model coefficients with corresponding 95% confidence intervals used to estimate AHI-LC_{AUTO} and RDTLC_{AUTO} respectively. In particular, Table 2 shows linear model coefficients used to estimate AHI-LC_{AUTO} and Table 3 shows linear model coefficients used to estimate RDI-LC_{AUTO}.

TABLE 2

| Model Coefficients | Mean | Confidence Interval (95%) |
|---|---|---|
| β₁ | −12.746 | [−12.799, −12.692] |
| β₂ | 0.389 | [0.387, 0.392] |
| β₃ | −195.198 | [−196.121, −194.276] |
| β₄ | 2.159 | [2.154, 2.164] |

TABLE 3

| Model Coefficients | Mean | Confidence Interval (95%) |
|---|---|---|
| β₁ | 3.133 | [3.080, 3.187] |
| β₂ | 0.565 | [0.563, 0.567] |
| β₃ | −276.584 | [−277.461, −275.706] |
| β₄ | 1.743 | [1.739, 1.747] |

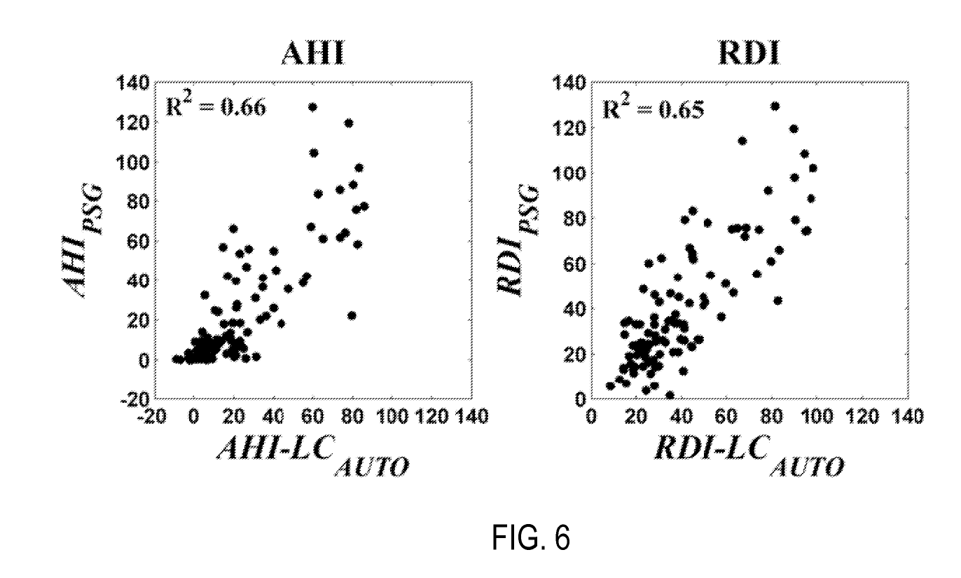

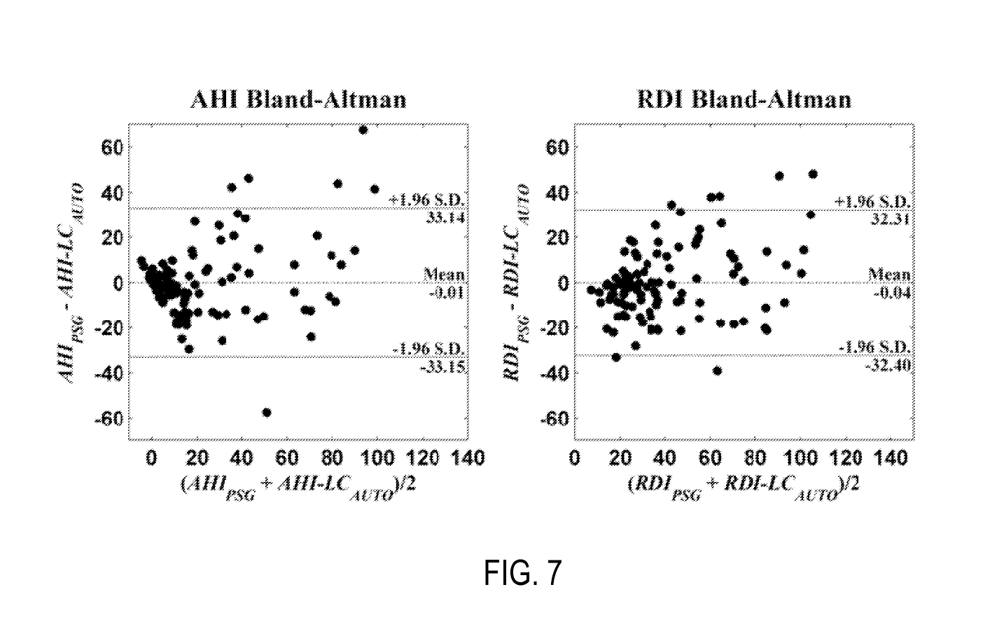

Direct comparison of AHI-LC_{AUTO} versus AHI-PSG and RDI-LC_{AUTO} versus RDI-PSG are shown in FIG. 6 with the corresponding R² values. Due to the regression analysis utilized to predict AHI-LC_{AUTO}, some predicted values ended up being negative. While a negative AHI is not traditionally logical, the negative AHI-LC_{AUTO} values only occurred for AHI-PSG values less than five suggesting that they are clinically equivalent to the absence of sleep apnea. Comparison of the difference between the automatic scoring of the load cell data and the PSG scoring are shown in the Bland-Altman plots in FIG. 7. In particular, FIG. 7 shows Bland-Altman plots showing the agreement between the AHI-PSG and AHI-LC_{AUTO} (left panel) and RDI-PSG and RDI-LC_{AUTO} (right panel).

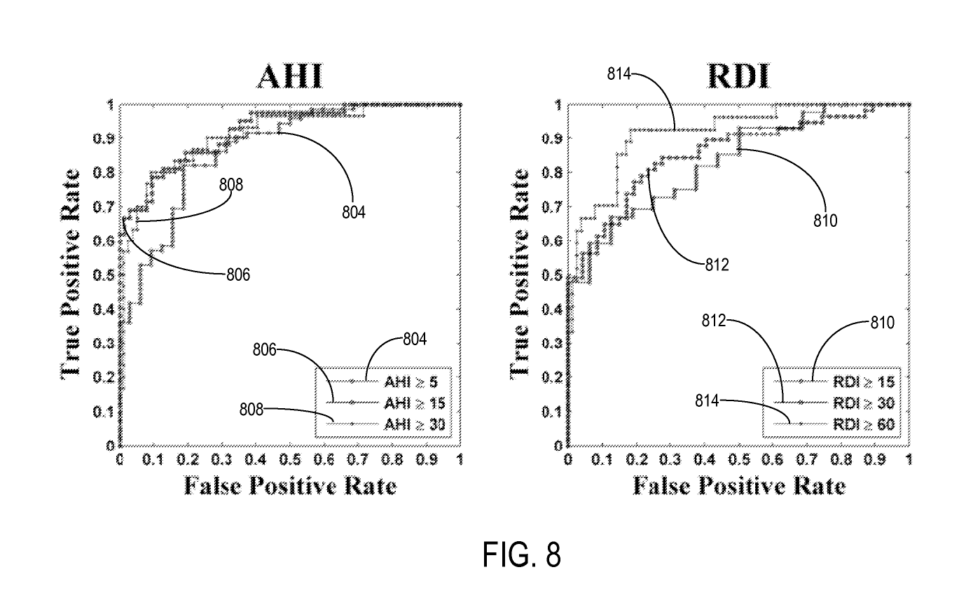

AHI-PSG was on average only 0.0058 less than AHI-LC_{AUTO}, which was not significant (t₁₀₃=−0.0035, p=0.9972 with a 95% confidence interval of [−3.2947, 3.2830]). RDI-PSG was on average 0.0449 less than that of RDI-LC_{AUTO}; this difference was also not significant (t₁₀₃=−0.0277, p=0.9780) with a 95% confidence interval of [−3.2556, 3.1659]). The receiver operating characteristic (ROC) curves showing the ability of the automatically scored load cell data to determine the presence of sleep apnea are shown in FIG. 8. In particular, FIG. 8 shows ROC curves showing the ability of the load cell data to predict overnight sleep studies positive for sleep apnea. The left plot displays the results for detecting sleep apnea at various thresholds of AHI-LC_{AUTO} when positive tests are defined as AHI-PSG≧5 (trace labeled 804), AHI-PSG≧15 (trace labeled 806), and AHI-PSG≧30 (trace labeled 808). The right plot displays the results for detecting sleep apnea at various thresholds of RDI-LC_{AUTO} when positive tests are defined as RDI-PSG≧15 (trace labeled 810), RDI-PSG≧30 (trace labeled 812), and RDI-PSG≧60 (trace labeled 814). The area under curve (AUC) for each cutoff level of AHI was 0.8698 for AHI-PSG≧5, 0.9220 for AHI-PSG≧15, and 0.9095 for AHI-PSG≧30. The AUC for each cutoff level of RDI was 0.8345 for RDI-PSG≧15, 0.8548 for RDI-PSG≧30, and 0.9173 for RDI-PSG≧60.

The algorithm described in this example illustrates an example method for using only load cell data to automatically detect sleep apnea. High AUC values from the ROC analysis (see FIG. 8) indicate that the load cell system has promise as a prescreening tool where high sensitivity is desired to confirm the suspicion of sleep apnea. There was some variability in the results; however, this is not surprising due to the high inconsistency that is seen in the visual scoring of PSG between different sleep technologists. Therefore, exact agreement between the AHI-LC_{AUTO} and RDI-LC_{AUTO} with AHI-PSG and RDI-PSG was not expected. The results of the automatic algorithm are especially encouraging considering that the load cell data was collected at two different sleep labs (OHSU and PSP). Good agreement despite the variability introduced by different sleep technologists scoring the overnight sleep tests and despite slightly different load cell setups (i.e., different beds with differing numbers of load cells under each bed) suggests robustness and generalizability in the system and the automatic algorithm.

The negative coefficient for the cV % arrived at in this example was also perplexing. It is counterintuitive that estimates for AHI-LC_{AUTO} or RDI-LC_{AUTO} would decrease with increasing variability in breathing amplitude as perceived by the cV % feature. The cV % feature was initially intended to capture the constant changes in breathing amplitude associated with recurring apneas and hypopneas. It may be that there is some complex interaction between the cV % feature and the DBI feature. It is possible that the DBI feature overestimates the presence of apneic and hypopneic events in individuals with high variability in their breathing amplitudes as detected in the CoP_{y} signal. In such a case, the cV % feature could act as some sort of compensation for this overestimation.

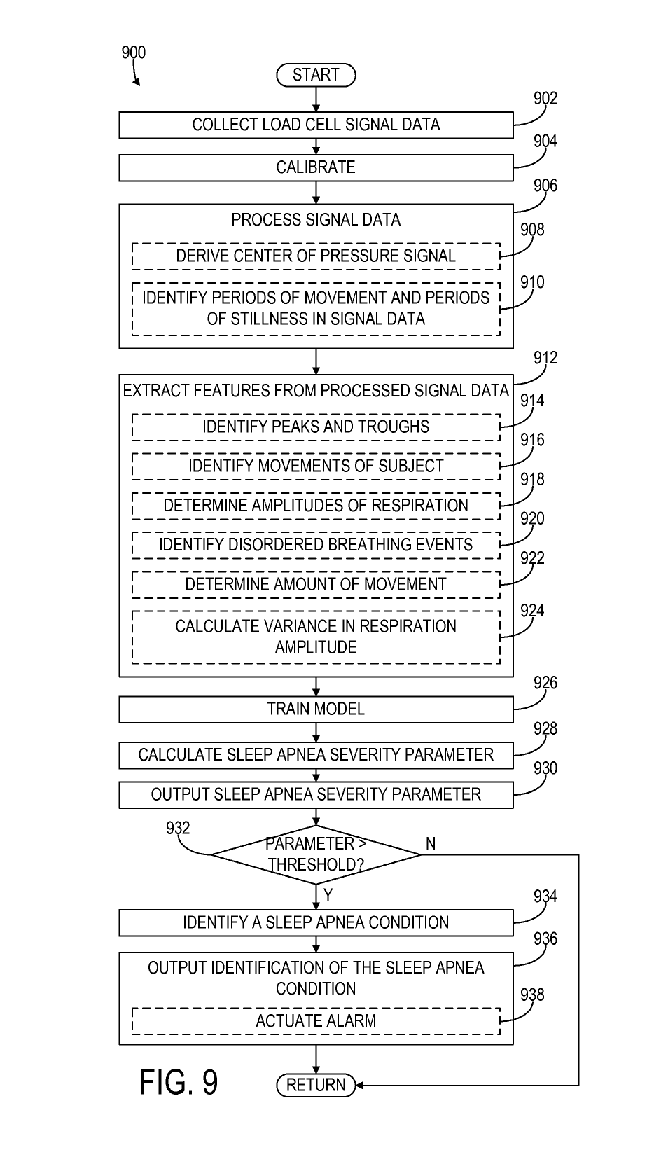

FIG. 9 shows an example method 900 for automatically identifying sleep apnea in a subject during sleep based on load cell signal data received from load cells coupled to or positioned beneath or between supports of a bed, such as shown in FIG. 1 described above. For example, a support for a bed may be configured to be added to the bed and tensioned for use with the load cell.

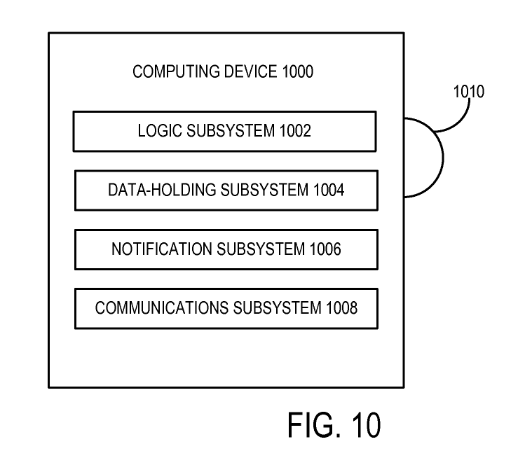

Method 900 may be executed via a computing device, such as the computing device described below with regard to FIG. 10. For example, one or more steps of method 900, such as the collecting, processing, extracting, calculating, and identifying steps, may be performed by a computing device comprising executable instructions for applying a model to features extracted from the signal data.

Further, in some examples, method 900 may be employed by an apparatus configured to receive information related to sleep apnea in a subject. In this example, the apparatus may comprise one or more load cells configured for placement below one or more supports of a bed such that the bed and the one or more bed supports are physically supported by the load cells, the load cells further configured to convert force to an electrical signal indicative of the force; and a computing device coupled to at least one of the one or more load cells, where the computing device comprises computer executable instructions for receiving signals from at least one of the one or more load cells. Such an apparatus may further comprise a transceiver coupled to at least one of the computing device and to at least one of the one or more load cells and/or an alarm. For example, the computer executable instructions may be operable to actuate an alarm in response to an identification of sleep apnea in the subject.

At 902, method 900 includes collecting load cell signal data. For example, load cell signal data may be continuously collected from one or more load cells for a duration. As remarked above, the load cells may be coupled to a bed in any suitable manner, e.g., positioned below one or more supports of a bed such that the bed and the one or more bed supports are physically supported by the load cells and the load cell signal data indicates force exerted against the load cell. Further, the duration during which load cell data is continuously collected may include both movement and stillness of the subject.

At 904, method 900 includes performing one or more calibration steps. For example, the load cell signal data may be calibrated based on a mass of the subject, based on physical characteristics of the bed and sensor system, based on an impulse response of the bed and sensor system, etc. For example, as remarked above, in some examples, after the load cells are installed to the bed, a calibration step may be performed on the bed/load cell system in order to characterize the response of the bed to movement so that processing of the load cell data may be adjusted and interpreted accordingly.

At 906, method 900 includes processing the signal data to obtain processed signal data. The signal data may be processed in any suitable way in order to condition the load cell data so that relevant features can be automatically extracted. For example, at 908, method 900 may include deriving a center of pressure signal from the signal data. For example, data from each load cell may be used to calculate a center of pressure along the head-to-toe axis of the bed as described above. As another example, at 910, method 900 may include identifying periods of movement and periods of stillness in the signal data, e.g., by identifying peaks and troughs in the signal data and automatically segmenting the data into regions corresponding movement and regions corresponding to stillness of the subject.

At 912, method 900 includes extracting features from the processed signal data. Features extracted from the processed signal data may include any suitable features used to assess sleep apnea severity via a suitable model. For example, extracting features from the processed signal data may comprise extracting the first, second, and third features as described above. For example, at 914, method 900 may include identifying peaks and troughs in the signal data throughout the duration. At 916, method 900 may include identifying movements of the subject throughout the duration based on the processed signal data. At 918, method 900 may include determining amplitudes of respiration of the subject throughout the duration based on the processed signal data, e.g., based on the identified peaks and troughs. In some examples, amplitudes of respiration may be estimated using Kalman filtering applied to the center of pressure signal in both the x and y directions. At 920, method 900 may include identifying disordered breathing events throughout the duration based on the amplitudes of respiration. At 920, method 900 may include determining an amount of movement throughout the duration based on the identified movements. For example, an amount of movement throughout the duration may be calculated via Equation 1 described above. At 924, method 900 may include calculating a variance in respiration amplitude based on the amplitudes of respiration throughout the duration. For example, the variance in respiration amplitude may be calculated as a ratio of time that a coefficient of variation for non-overlapping windows of the processed signal data is above a predetermined threshold, e.g., as described above with regard to Equation 3. In some examples, a duration of each non-overlapping window may be approximately five seconds. As other examples, extracting features from the processed signal data may include performing a frequency analysis on the data, e.g., to identify a power difference in breathing frequencies between apneic and non-apneic segments. Such power differences may be used to estimate “normal” breathing amplitudes so that deviations from these normal breathing amplitudes may be more readily detected.

At 926, method 900 includes training a model. For example, a linear model with constant coefficients such as described above with regard to Equation 5 may be trained on clinically estimated sleep apnea severity data. For example, the constant coefficients of the linear model, such as the model described above with regard to Equation 5, may be estimated based on training data. At 928, method 900 includes calculating a sleep apnea severity parameter based on the extracted features via the model. For example, the sleep apnea severity parameter may be determined via Equation 5 described above.

At 930, method 900 may include outputting the sleep apnea severity parameter. In some examples, outputting the sleep apnea severity parameter may comprise outputting the parameter to a display device and/or an audio device (e.g., one or more speakers), sending the parameters to a remote computing device over a network, storing the parameter in a memory component of a computing device, etc. For example, as remarked above, in embodiments, a computing device may be adapted to send data such as the sleep apnea severity parameter to an external computing device to communicate information about a subject's sleep.

At 932, method 900 includes determining if the sleep apnea severity parameter is greater than a threshold. If the sleep apnea parameter is greater than a threshold, method 900 proceeds to 934. At 934, method 900 includes identifying a sleep apnea condition. For example, a flag may be set in a memory component of a computing device indicating that a sleep apnea condition is present. At 936, method 900 includes outputting an identification of the sleep apnea condition. For example, the identification of the sleep apnea condition may be output to a display device, output to an audio device, stored in a memory component of a computing device, sent to a remote computing device via a network, etc. In some examples, a notification may be performed in response to the identification of a sleep apnea condition which persists for a time greater than a predetermined threshold, e.g., a long central apnea condition. For example, at 938, method 900 may include actuating an alarm in order to alert the subject or a clinician of the presence of a sleep apnea condition where the subject has stopped breathing for a duration greater than a predetermined threshold time duration.

In some embodiments, the above described methods and processes may be tied to a computing system, such as computing device 130 shown in FIG. 1, including one or more computers. In particular, the methods and processes described herein, e.g., method 900 described above, may be implemented as a computer application, computer service, computer API, computer library, and/or other computer program product.

FIG. 10 schematically shows a nonlimiting computing device 1000 that may perform one or more of the above described methods and processes. Computing device 1000 is shown in simplified form. It is to be understood that virtually any computer architecture may be used without departing from the scope of this disclosure. In different embodiments, computing device 1000 may take the form of a microcomputer, an integrated computer circuit, microchip, a mainframe computer, server computer, desktop computer, laptop computer, tablet computer, home entertainment computer, network computing device, mobile computing device, mobile communication device, gaming device, etc.

Computing device 1000 includes a logic subsystem 1002 and a data-holding subsystem 1004. Computing device 1000 may optionally include a notification subsystem 1006 and a communication subsystem 1008, and/or other components not shown in FIG. 10. Computing device 1000 may also optionally include user input devices such as manually actuated buttons, switches, keyboards, mice, game controllers, cameras, microphones, and/or touch screens, for example.

Logic subsystem 1002 may include one or more physical devices configured to execute one or more machine-readable instructions. For example, the logic subsystem may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

The logic subsystem may include one or more processors that are configured to execute software instructions. Additionally or alternatively, the logic subsystem may include one or more hardware or firmware logic machines configured to execute hardware or firmware instructions. Processors of the logic subsystem may be single core or multicore, and the programs executed thereon may be configured for parallel or distributed processing. The logic subsystem may optionally include individual components that are distributed throughout two or more devices, which may be remotely located and/or configured for coordinated processing. One or more aspects of the logic subsystem may be virtualized and executed by remotely accessible networked computing devices configured in a cloud computing configuration.

Data-holding subsystem 1004 may include one or more physical, non-transitory, devices configured to hold data and/or instructions executable by the logic subsystem to implement the herein described methods and processes. When such methods and processes are implemented, the state of data-holding subsystem 1004 may be transformed (e.g., to hold different data).

Data-holding subsystem 1004 may include removable media and/or built-in devices. Data-holding subsystem 1004 may include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 1004 may include devices with one or more of the following characteristics: volatile, nonvolatile, dynamic, static, read/write, read-only, random access, sequential access, location addressable, file addressable, and content addressable. In some embodiments, logic subsystem 1002 and data-holding subsystem 1004 may be integrated into one or more common devices, such as an application specific integrated circuit or a system on a chip.

FIG. 10 also shows an aspect of the data-holding subsystem in the form of removable computer-readable storage media 1010, which may be used to store and/or transfer data and/or instructions executable to implement the herein described methods and processes. Removable computer-readable storage media 1010 may take the form of CDs, DVDs, HD-DVDs, Blu-Ray Discs, EEPROMs, flash memory cards, and/or floppy disks, among others.

When included, notification subsystem 1006 may be used to present visual and/or audio and/or haptic representations of data held by data-holding subsystem 1004. For example, notification subsystem 1006 may be used to present indications of sleep apnea conditions to a subject and/or a clinician. As the herein described methods and processes change the data held by the data-holding subsystem, and thus transform the state of the data-holding subsystem, the state of notification subsystem 1006 may likewise be transformed to visually and/or sonically and/or haptically represent changes in the underlying data. Notification subsystem 1006 may include one or more display devices utilizing virtually any type of technology. Such display devices may be combined with logic subsystem 1002 and/or data-holding subsystem 1004 in a shared enclosure, or such display devices may be peripheral display devices. Notification subsystem 1006 may include one or more audio devices, e.g., one or more speakers, and/or one or more haptic devices utilizing virtually any type of technology.

When included, communication subsystem 1008 may be configured to communicatively couple computing device 1000 with one or more other computing devices. Communication subsystem 1008 may include wired and/or wireless communication devices compatible with one or more different communication protocols. As nonlimiting examples, the communication subsystem may be configured for communication via a wireless telephone network, a wireless local area network, a wired local area network, a wireless wide area network, a wired wide area network, etc. In some embodiments, the communication subsystem may allow computing device 1000 to send and/or receive messages to and/or from other devices via a network such as the Internet. For example, communication subsystem 1008 may allow sleep analysis data derived from load cell signal data to be sent to and/or from other devices via a network.

It is to be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated may be performed in the sequence illustrated, in other sequences, in parallel, or in some cases omitted. Likewise, the order of the above-described processes may be changed.

The subject matter of the present disclosure includes all novel and nonobvious combinations and subcombinations of the various processes, systems and configurations, and other features, functions, acts, and/or properties disclosed herein, as well as any and all equivalents thereof.

1. A method for automatically identifying sleep apnea in a subject during sleep, the method comprising:
continuously collecting load cell signal data from one or more load cells for a duration, the load cells coupled to one or more supports of a bed such that the load cell signal data indicates force exerted against the load cell;
processing the signal data to obtain processed signal data;
extracting features from the processed signal data;
calculating a sleep apnea severity parameter based on the extracted features via a model; and
identifying sleep apnea in the subject based on the sleep apnea severity parameter;
wherein the collecting, processing, extracting, calculating, and identifying are performed by a computing device comprising executable instructions for applying the model to features extracted from the signal data.

2. The method of claim 1, wherein the duration includes movement and stillness of the subject.

3. The method of claim 1, wherein processing the signal data to obtain processed signal data comprises deriving a center of pressure signal from the signal data.

4. The method of claim 1, further comprising calibrating the load cell signal data based on a mass of the subject.

5. The method of claim 1, wherein extracting features from the processed signal data comprises identifying movements of the subject throughout the duration based on the processed signal data and determining amplitudes of respiration of the subject throughout the duration based on the processed signal data.

6. The method of claim 5, wherein extracting features from the processed signal data further comprises identifying peaks and troughs in the signal data throughout the duration and determining amplitudes of respiration of the subject throughout the duration based on the identified peaks and troughs.

7. The method of claim 5, wherein extracting features from the processed signal data further comprises identifying disordered breathing events throughout the duration based on the amplitudes of respiration, determining an amount of movement throughout the duration based on the identified movements, and calculating a variance in respiration amplitude based on the amplitudes of respiration throughout the duration.

8. The method of claim 7, wherein the variance in respiration amplitude is calculated as a ratio of time that a coefficient of variation for non-overlapping windows of the processed signal data is above a predetermined threshold.

9. The method of claim 8, wherein a duration of each non-overlapping window is approximately five seconds.

10. The method of claim 7, wherein the sleep apnea severity parameter is determined via the equation
β₁+β₂(MI)+β₃(cV %)+β₄(DBI),
where MI is the amount of movement throughout the duration, cV % is the variance in respiration amplitude, DBI is the number of identified disordered breathing events throughout the duration that meet a predetermined minimum time duration constraint, and β₁, β₂, β₃, and β₄ are constant coefficients.

11. The method of claim 10, wherein β₁, β₂, β₃, and β₄ are estimated using linear regression applied to training data.

12. An apparatus configured to receive information related to sleep apnea in a subject, the apparatus comprising:
one or more load cells configured for placement below one or more supports of a bed such that the bed and the one or more bed supports are physically supported by the load cells, the load cells further configured to convert force to an electrical signal indicative of the force; and
a computing device coupled to at least one of the one or more load cells, the computing device comprising computer executable instructions for receiving signals from at least one of the one or more load cells,
processing the signals to obtain signals representing periods of movement and signals representing periods of stillness,
extracting features from the signals representing periods of movement and the signals representing periods of stillness,
calculating a sleep apnea severity parameter based on the extracted features via a model, and
identifying sleep apnea in the subject based on the sleep apnea severity parameter.

13. The apparatus of claim 12, further comprising a transceiver coupled to at least one of the computing device and to at least one of the one or more load cells.

14. The apparatus of claim 12, further comprising an alarm, the computer executable instructions operable to actuate the alarm in response to identifying sleep apnea in the subject.

15. The apparatus of claim 12, wherein identifying sleep apnea in the subject based on the sleep apnea severity parameter comprises identifying sleep apnea in response to the sleep apnea parameter greater than a predetermined threshold.

16. The apparatus of claim 12, further comprising a support for a bed, the support configured to be added to the bed and tensioned for use with the load cell.

17. The apparatus of claim 12, wherein the model is a linear model with constant coefficients.

18. The apparatus of claim 17, wherein the linear model is trained on clinically estimated sleep apnea severity.

19. A method for automatically identifying sleep apnea in a subject during sleep, the method comprising:
continuously collecting load cell signal data from one or more load cells for a duration, the duration including movement of the subject and stillness of the subject, the load cells being positioned below one or more supports of a bed such that the bed and the one or more bed supports are physically supported by the load cells and the load cell signal data indicates force exerted against the load cell;
processing the signal data to obtain processed signal data;
identifying movements of the subject throughout the duration based on the processed signal data;
determining amplitudes of respiration of the subject throughout the duration based on the processed signal data;
identifying disordered breathing events throughout the duration based on the amplitudes of respiration;
determining an amount of movement throughout the duration based on the identified movements;
calculating a variance in respiration amplitude based on the amplitudes of respiration throughout the duration;
calculating a sleep apnea severity parameter based on the number of identified disordered breathing events throughout the duration that meet a predetermined minimum time duration constraint, the amount of movement throughout the duration, and the variance in respiration amplitude; and
in response to the sleep apnea severity parameter greater than a threshold, identifying sleep apnea in the subject;
wherein the collecting, processing, determining, calculating, and identifying are performed by a computing device.

20. The method of claim 19, wherein the sleep apnea severity parameter is determined via a linear model with constant coefficients, where the constant coefficients are estimated based on training data.
